(19) United States
(12) Patent Application Publication
(10) Pub. No.: US 2024/0407707 A1
(43) Pub. Date: Dec. 12, 2024

(54) Sensor Mouthguard Device for Detecting Brain Injuries

(71) Applicant: Louis Eke, Ashburn, VA (US)

(72) Inventor: Louis Eke, Ashburn, VA (US)

(21) Appl. No.: 18/738,160

(22) Filed: Jun. 10, 2024

Related U.S. Application Data
(60) Provisional application No. 63/507,529, filed on Jun. 12, 2023.

(51) Int. Cl. A61B 5/369; A61B 5/00

(52) U.S. Cl. CPC A61B 5/369; A61B 5/0022; A61B 5/682; A61B 2562/0219

(57) ABSTRACT

A sensor mouthguard device comprising an upper and lower silicone mouthpiece accommodating flex circuits embedded with accelerometers, biosensors, a microprocessor, and computing modules is disclosed. The device includes a Bluetooth module for real-time data transmission to remote computing devices, facilitating immediate clinical evaluation. The device automatically activates upon placement in the mouth and measures linear and angular acceleration forces and brain wave activity using Quantitative Electroencephalogram (QEEG) sensors. The device includes a replaceable and rechargeable lithium battery, and also includes a long-range antenna for extended communication capabilities. The upper and lower silicone mouthpiece are coupled using a pair of couplers for enabling movement of the mouthguard device when the jaw is moved by a user.

### CROSS-REFERENCE TO RELATED APPLICATION

The present application claims priority to, and the benefit of, U.S. Provisional Application No. 63/507,529, which was filed on Jun. 12, 2023, and is incorporated herein by reference in its entirety.

### FIELD OF THE INVENTION

The present invention generally relates to the field of medical devices. More specifically, the present invention relates to a novel mouthguard device (i.e., malleable neurosensory mouthguard) designed to fit in the mouths of individuals susceptible to activities or conditions leading to traumatic brain injuries or chronic brain dysfunctions. The device includes sensors to track location and severity of a brain injury and the information can be sent to a programmed wireless device in real time for early clinical intervention. Accordingly, the present disclosure makes specific reference thereto. Nonetheless, it is to be appreciated that aspects of the present invention are also equally applicable to other like applications, devices, and methods of manufacture.

### BACKGROUND

By way of background, contact sports athletes and military combat personnel are frequently exposed to head-on collisions or combat blasts which can lead to varying degrees of traumatic brain injury (TBI), such as concussions or posttraumatic stress disorder (PTSD). These injuries can be challenging to diagnose accurately, as the nature and extent of the damage are not easily measurable. Although nearly 90% of these injuries may resolve without long-term effects, some cases may progress to a severe and progressive brain condition known as chronic traumatic encephalopathy (CTE).

One of the common challenges faced by athletes and combat personnel is the inability to obtain accurate information about prior impact injuries or the degenerating state of their brain cells or cognitive dysfunction. The lack of precise data makes it difficult for individuals to understand the full impact of their injuries and the current health of their brain. Additionally, due to the absence of objective and accurate information, athletes and military personnel often face pressure to return to their activities prematurely. The pressure to continue participating without adequate recovery poses significant risks to their long-term health.

The issue is further exacerbated by the high error rate in diagnosing and treating TBIs. The symptoms of brain injuries and cognitive dysfunctions are often generalized and can be mistaken for other conditions, leading to misdiagnosis and inadequate treatment. Existing methods do not provide real time analysis and do not help in quick diagnosis. Therefore, there is a need for a reliable method and device to objectively measure and monitor brain injuries and/or chronic neurological dysfunction. Further, individuals with cognitive neurological dysfunction, or chronic neurological disease (or potential therefore), including chronic brain conditions related to strokes, aneurysms, ALZ, and dementia need a medical device that can detect and measure brain related conditions/dysfunctions/degradations. The device can be inserted into a user's mouth for triggering an autonomous neuro activity tracking, chemical synthesis, reporting and predicting, and diagnosing brain conditions.

Therefore, there exists a long felt need in the art for a medical device that can effectively measure linear and angular (rotational) head impacts to the brain caused by contact sports, blasts, or collisions. There is also a long felt need in the art for an innovative mouthguard device that can be easily worn by athletes and military combat personnel to detect linear and angular head impacts. Additionally, there is a long felt need in the art for a mouthguard device that accurately determines the location and severity of an injury. Moreover, there is a long felt need in the art for a uniquely designed sensor mouthguard device (i.e., malleable neurosensory mouthguard) that can transmit sensor data in real-time via Bluetooth to a programmed wireless device for early clinical intervention. Further, there is a long felt need in the art for a mouthguard device that improves the safety and health outcomes for athletes and military personnel. Finally, there is a long felt need in the art for a medical device that can be used inside the mouth by a user for providing biochemical information about their chronic brain conditions/dysfunctions/degradations and ensuring appropriate treatment and recovery time.

The subject matter disclosed and claimed herein, in one embodiment thereof, comprises a sensor mouthguard device (i.e., malleable neurosensory mouthguard) for monitoring head impacts and injuries. The device features an upper silicone mouthpiece and adapted to accommodate a top flex circuit therein, the top flex circuit is equipped with at least one accelerometer for measuring acceleration forces and at least one biosensor for measuring brain wave activity, a Bluetooth module disposed on the top flex circuit for real-time communication with a remote electronic computing device, a lower silicone mouthpiece designed to accommodate a lower flex circuit therein, the lower flex circuit is equipped with a microprocessor and multiple computing modules for processing sensor data, a second biosensor for additional brain wave measurement, and a long-range antenna for extending the communication range, a coupler mechanism pivotally connects the upper and lower silicone mouthpieces, enabling movement in coordination with jaw movements. The lower mouthpiece portion having added biochemical nano chemical sensors for absorbing saliva and blood traces into a chemical test-absorbent material which can synthesize ion proteins (i.e., brain chemical proteins) and detect brain tissue abnormalities. The nano micro-chemical synthesizer processes the test and generates a predictive result analysis for further laboratory testing and intervention.

In this manner, the sensor mouthguard device (i.e., malleable neurosensory mouthguard) of the present invention accomplishes all of the forgoing objectives and provides users with a novel mouthguard device that provides precise measurements of both linear and angular forces experienced by the head during impacts, ensuring a comprehensive understanding of the injury. The device performs real-time monitoring of brain wave activity to provide detailed insights into the brain's condition following an impact. The mouthguard automatically activates upon being placed in the mouth thereby providing easy use. The mouthguard can wirelessly communicate with various electronic devices, such as smartphones, tablets, and laptops, facilitating easy access to the data and integration with existing health monitoring systems. The mouthguard device is useful for contact sports athletes and military personnel by continuously monitoring and reporting head impact data, thereby contributing to better injury management and prevention strategies.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present general some concepts in a simplified form as a prelude to the more detailed description that is presented later.

The subject matter disclosed and claimed herein, in one embodiment thereof, comprises a sensor mouthguard device (i.e., malleable neurosensory mouthguard) for monitoring and imaging head impacts. The device comprises an upper silicone mouthpiece anatomically designed to resemble an upper jaw and adapted to accommodate a top flex circuit therein, the top flex circuit is equipped with at least one accelerometer for measuring acceleration forces and at least one biosensor for measuring brain wave activity, a Bluetooth module disposed on the top flex circuit for real-time communication with a remote electronic computing device, lower silicone mouthpiece designed to accommodate a lower flex circuit therein, the lower flex circuit is equipped with a microprocessor and multiple computing modules for processing sensor data, a second biosensor for brain wave measurement, and a long-range antenna for extending the communication range, a coupler mechanism pivotally connects the upper and lower silicone mouthpieces, enabling movement in coordination with jaw movements. The lower mouthpiece portion having added biochemical nano chemical sensors for absorbing saliva and blood traces into a chemical test-absorbent material which can synthesize ion proteins (i.e., brain chemical proteins) and detect brain tissue abnormalities. The nano micro-chemical synthesizer processes the test and generates a predictive result analysis for further laboratory testing and intervention.

In yet another embodiment, the device includes a power module in the form of a replaceable lithium battery for providing electrical power to the device, wherein the device automatically activates upon placement in the mouth and is configured to wirelessly transmit data for clinical intervention.

In another embodiment, the biosensor comprises a Quantitative Electroencephalogram (QEEG) adapted to monitor brain wave activity in real-time.

In another aspect, the device includes an Inertial Measurement Unit (IMU) chip disposed on the top flex circuit for additional measurement of angular and linear forces.

In yet another aspect, the device comprises an infrared-based obstacle detection device disposed on the lower flex circuit for detecting obstacles during use.

In still another embodiment, a method for monitoring and imaging head impacts using a sensor mouthguard device (i.e., malleable neurosensory mouthguard) is disclosed. The method includes providing a sensor mouthguard device, the mouthguard device includes an upper silicone mouthpiece designed to accommodate a top flex circuit therein, the top flex circuit equipped with at least one accelerometer and at least one biosensor for measuring brain wave activity, a lower silicone mouthpiece designed to accommodate a lower flex circuit therein, the lower flex circuit equipped with a microprocessor, multiple computing modules, and a second biosensor for additional brain wave measurement, a Bluetooth module for wireless communication, a power module including a replaceable lithium battery and a coupler mechanism for pivotally connecting the upper and lower silicone mouthpieces. The method also includes placing the sensor mouthguard device (i.e., malleable neurosensory mouthguard) in the mouth of a user to automatically activate the device, measuring linear and angular acceleration forces experienced by the user's head during an impact using the accelerometer, monitoring brain wave activity of the user in real-time using the biosensors, processing the measured acceleration and brain wave data with the microprocessor and computing modules, and transmitting the processed data wirelessly via the Bluetooth module to a remote electronic computing device.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of the malleable neurosensory mouthguard device of the present invention with detached battery and cover in accordance with the disclosed structure;

FIG. 1B illustrates another perspective view of the malleable neurosensory mouthguard device of the present invention with detached battery and cover in accordance with one embodiment of the disclosed structure;

FIG. 2 illustrates a perspective view of the malleable neurosensory mouthguard device of the present invention in a closed state in accordance with one embodiment of the disclosed structure;

FIG. 3A illustrates a perspective view of the top flex circuit of the mouthguard device of the present invention in accordance with the disclosed structure;

FIG. 3B illustrates another perspective view of the top flex circuit of the mouthguard device of the present invention in accordance with the disclosed structure;

FIG. 3C illustrates a top perspective view of the top flex circuit of the mouthguard device of the present invention in accordance with the disclosed structure;

FIG. 4A illustrates a perspective view of the lower flex circuit of the mouthguard device of the present invention in accordance with the disclosed structure;

FIG. 4B illustrates another perspective view of the lower flex circuit of the mouthguard device of the present invention in accordance with the disclosed structure;

FIG. 5 illustrates a perspective view of an exemplary coupler for coupling the upper mouthpiece and lower mouthpiece of the mouthguard device of the present invention in accordance with the disclosed structure;

FIG. 6A illustrates a perspective view of the lower mouthpiece of the mouthguard device of the present invention in accordance with the disclosed structure;

FIG. 6B illustrates a top perspective view of the lower mouthpiece of the mouthguard device of the present invention in accordance with the disclosed structure;

FIG. 7 illustrates a perspective view of the upper mouthpiece of the mouthguard device of the present invention in accordance with the disclosed structure;

FIG. 8 illustrates an isolated view of the long-range antenna used in the mouthguard device of the present invention in accordance with the disclosed structure;

FIG. 9 illustrates isolated views of the coin battery used in the mouthguard device of the present invention in accordance with the disclosed structure;

FIG. 10 illustrates a perspective of the coin battery bracket in accordance with the disclosed structure;

FIG. 11 illustrates an exploded view of the malleable neurosensory mouthguard device of the present invention in accordance with the disclosed structure;

FIG. 12 illustrates a front view of the malleable neurosensory mouthguard device of the present invention in the closed state in accordance with the disclosed structure;

FIG. 13 illustrates a front view of the malleable neurosensory mouthguard device of the present invention in the open state in accordance with the disclosed structure;

FIG. 14 illustrates a side perspective view of the malleable neurosensory mouthguard device of the present invention in the open state in accordance with the disclosed structure; and

FIG. 15 illustrates a schematic view showing communication between the mouthpiece device and a computing device.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a exists a long felt need in the art for medical device that can effectively measure linear and angular (rotational) head impacts to the brain caused by contact sports, blasts, or collisions. There is also a long felt need in the art for an innovative mouthguard device that can be easily worn by athletes and military combat personnel to detect linear and angular head impacts. Additionally, there is a long felt need in the art for a mouthguard device that accurately determines the location and severity of an injury. Moreover, there is a long felt need in the art for a uniquely designed sensor mouthguard device (i.e., malleable neurosensory mouthguard) that can transmit sensor data in real-time via Bluetooth to a programmed wireless device for early clinical intervention. Further, there is a long felt need in the art for a mouthguard device that improves the safety and health outcomes for athletes and military personnel. Finally, there is a long felt need in the art for a medical device that can be used inside the mouth by a user for providing biochemical information about their chronic brain conditions/dysfunctions/degradations and ensuring appropriate treatment and recovery time.

The present invention, in one exemplary embodiment, is a sensor mouthguard device (i.e., malleable neurosensory mouthguard) for monitoring and imaging head impacts. The device comprises an upper silicone mouthpiece, a top flex circuit, the top flex circuit is equipped with at least one accelerometer for measuring acceleration forces and at least one biosensor for measuring brain wave activity, a Bluetooth module for real-time communication with a remote electronic computing device, a lower silicone mouthpiece to accommodate a lower flex circuit therein, the lower flex circuit is equipped with a microprocessor and multiple computing modules for processing sensor data, a second biosensor for additional brain wave measurement, and a long-range antenna for extending the communication range. A pair of couplers pivotally connects the upper and lower silicone mouthpieces, enabling movement in coordination with jaw movements.

Reference will now be made in detail to the present preferred embodiments of the invention, examples of which are illustrated in the accompanying drawings. Wherever possible, the same reference numerals are used in the drawings and the description to refer to the same or like parts.

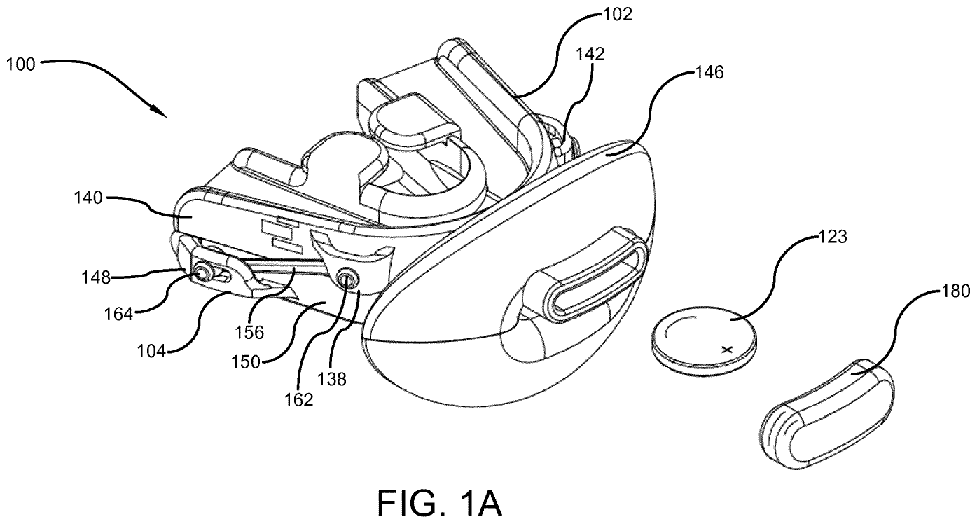

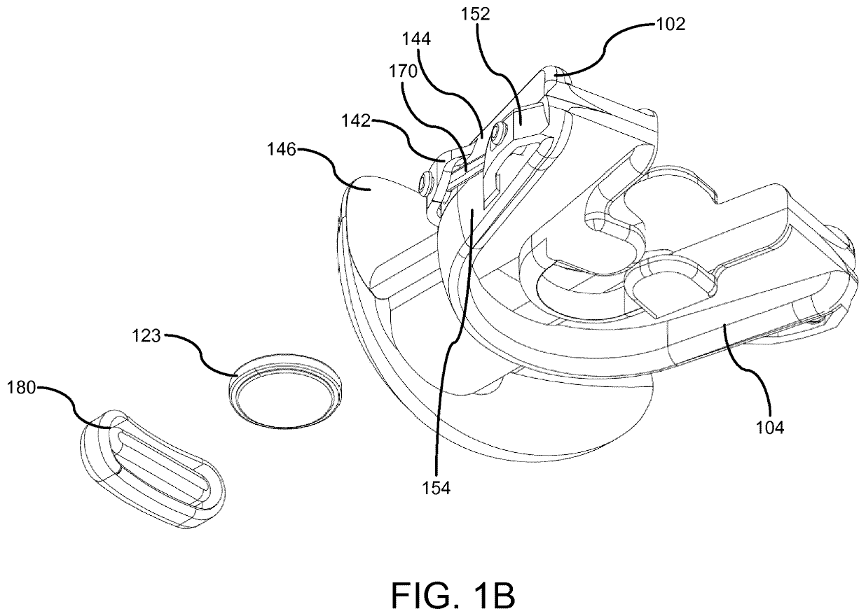

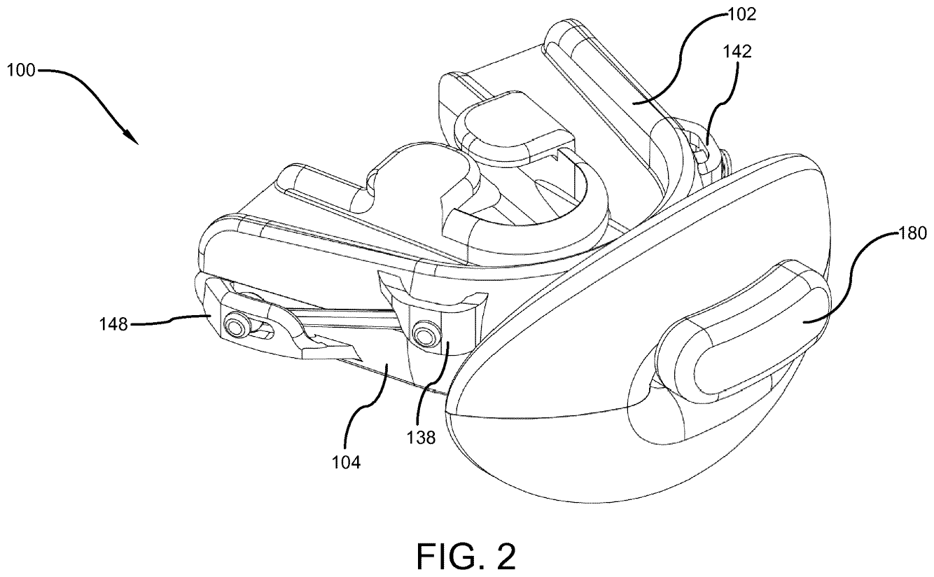

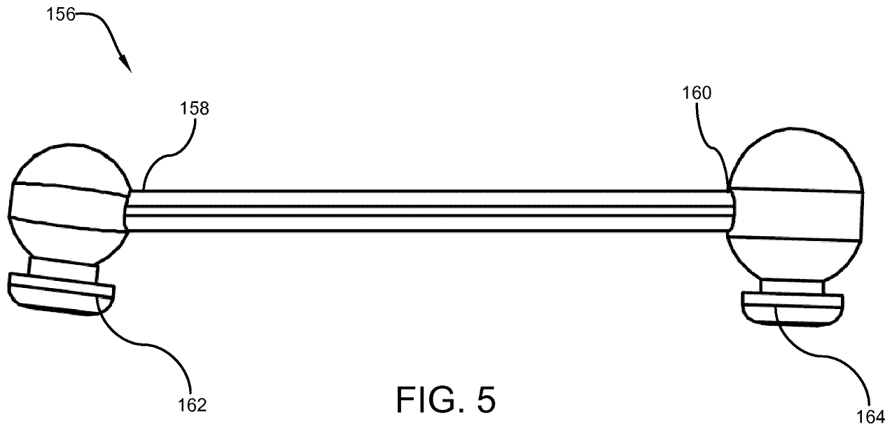

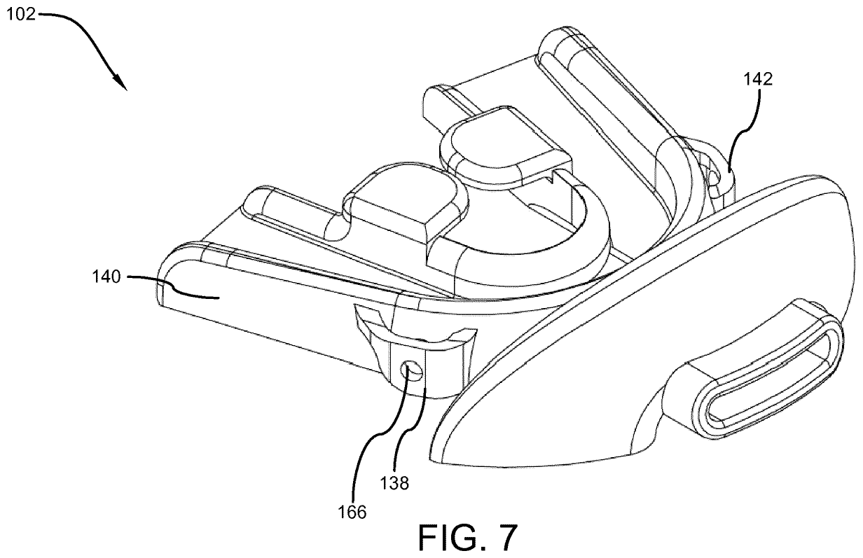

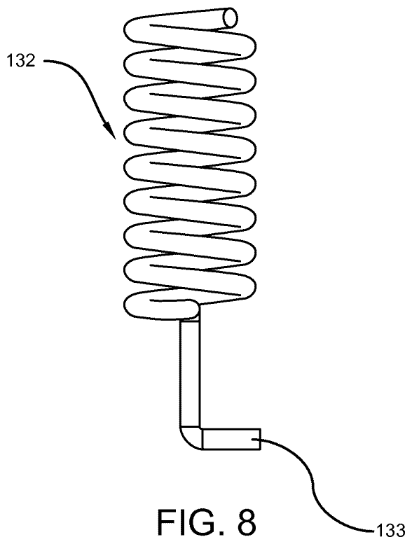

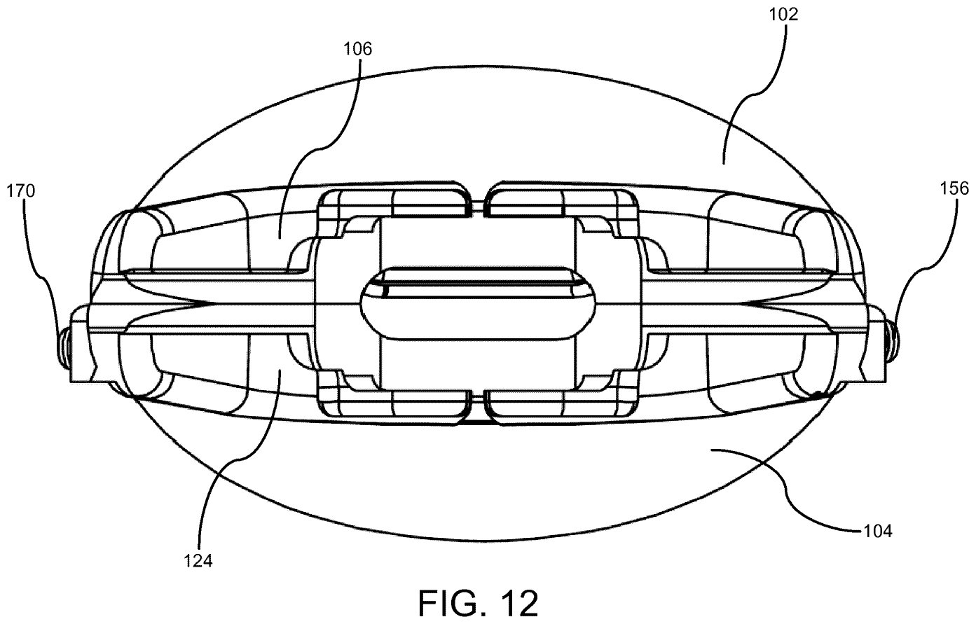

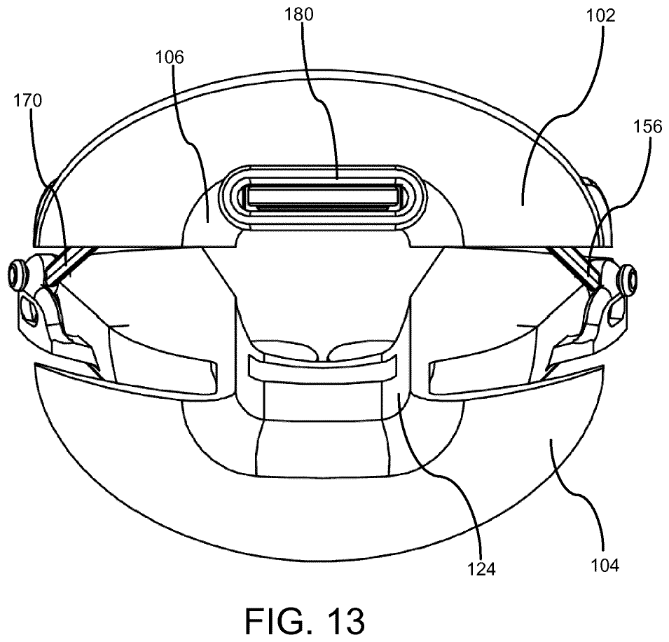

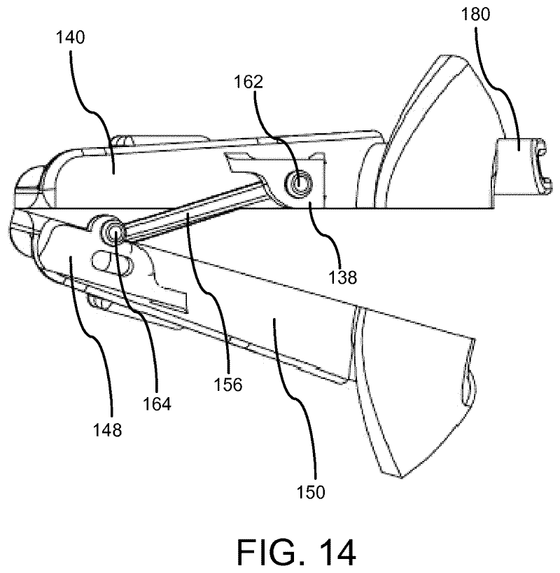

Referring initially to the drawings, FIGS. 1-14 illustrate different perspective views of malleable neurosensory mouthguard device of the present invention in accordance with the disclosed structure. The malleable neurosensory mouthguard device 100 is designed to provide accurate monitoring and imaging of head impacts, particularly useful for contact sports athletes and military personnel. The mouthguard serves as a clinical tool to objectively image and track head impact injuries and measures linear and angular (rotational) impacts to the brain caused by contact sports, blasts, or collisions. The mouthguard 100 is adapted to synthesize gamma wave and is automatically activated when the mouthguard is firmly placed in the mouth.

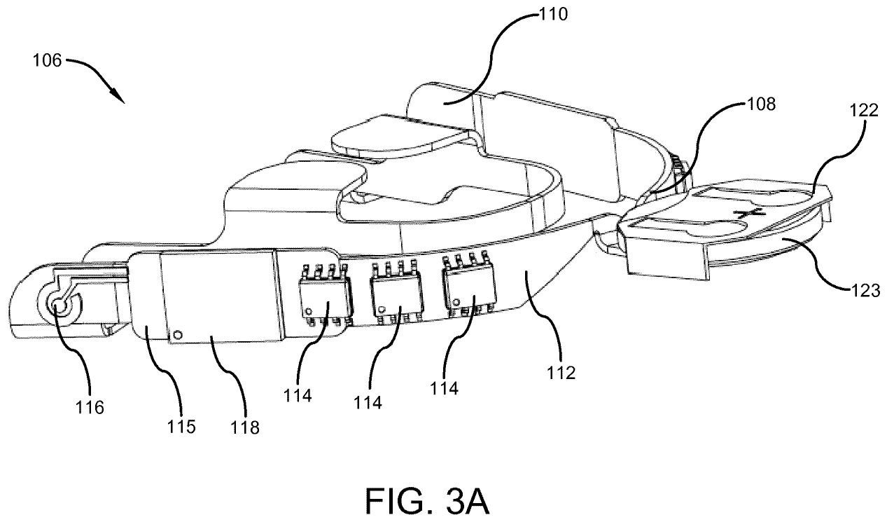

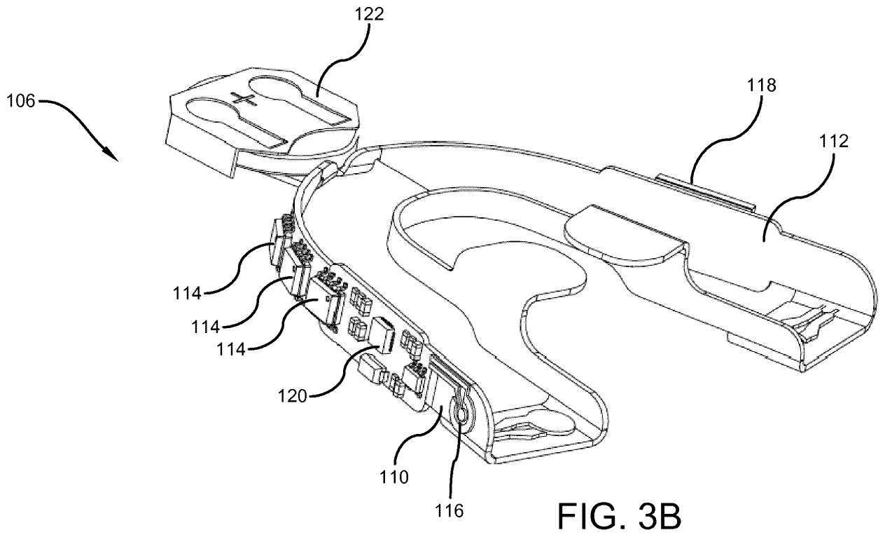

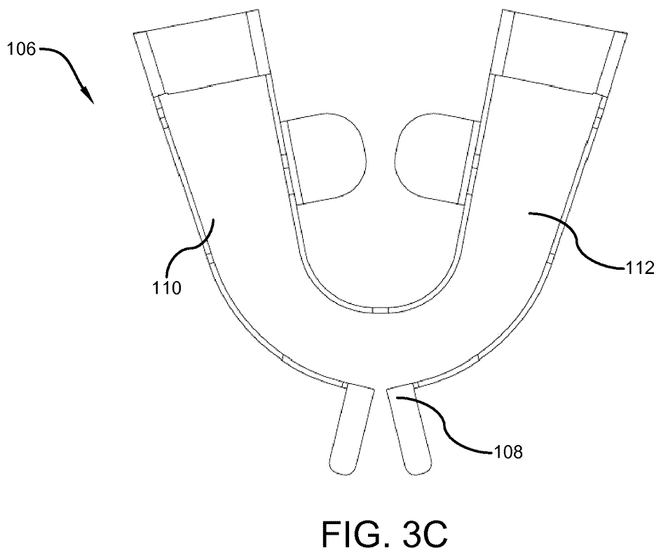

Referring to FIGS. 1A-4B, the mouthguard device 100 includes an upper silicone or nylopolyethine material mouthpiece 102 and a lower silicone or nylopolyethine material mouthpiece 104. The upper silicone mouthpiece 102 is designed to be anatomically similar (i.e., conforming) to upper jaw of a user and is adapted to accommodate a top flex circuit 106 embedded therein. Referring to FIGS. 3A-3C, the top flex circuit 106 includes a proximal end 108 and two sides 110, 112 extends therefrom for forming the jaw-shaped structure. The top flex circuit 106 has the same shape, size, and design as the upper silicone mouthpiece 102 and includes at least one accelerometer or accelerator 114 disposed thereon on the side 112. The accelerometer or accelerator 114 can be embedded in the outer surface 115 of the top flex circuit 106. The accelerometer or accelerator 114 is adapted to measure the acceleration forces experienced by the head of wearer of the device 100 during an impact. In one exemplary embodiment, the accelerometer 114 is a triaxial accelerometer. The top flex circuit 106 also includes at least one first biosensor 116 thereon. Biosensor 116 is adapted to include embedded Electroencephalogram (EEG) to measure EEG of the user using the device 100 in real-time. The EEG is preferably Quantitative Electroencephalogram (QEEG) and is adapted to monitor brain wave activity of wearer of the device 100.

A Bluetooth module 118 is disposed on the top flex circuit 106 and enables real-time communication between the mouthguard device 100 and a remote electronic computing device such as a smartphone, laptop, and the like. An inertial measurement unit (IMU) chip 120 is also disposed on the outer surface 115 of the top flex circuit 106. On the proximal end 108 of the top flex circuit 106, a replaceable bracket 122 for a lithium battery 123 is disposed which is configured to removably store the replaceable lithium battery for providing electrical power for the functioning of the device 100.

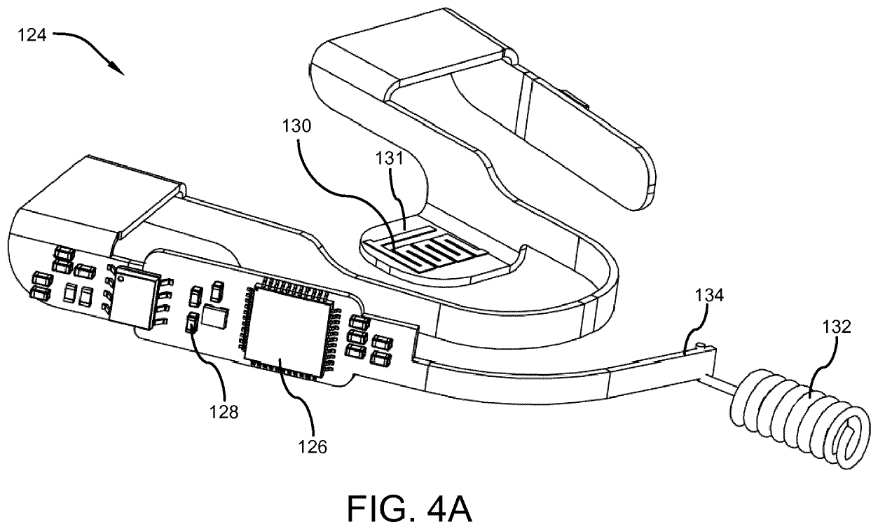

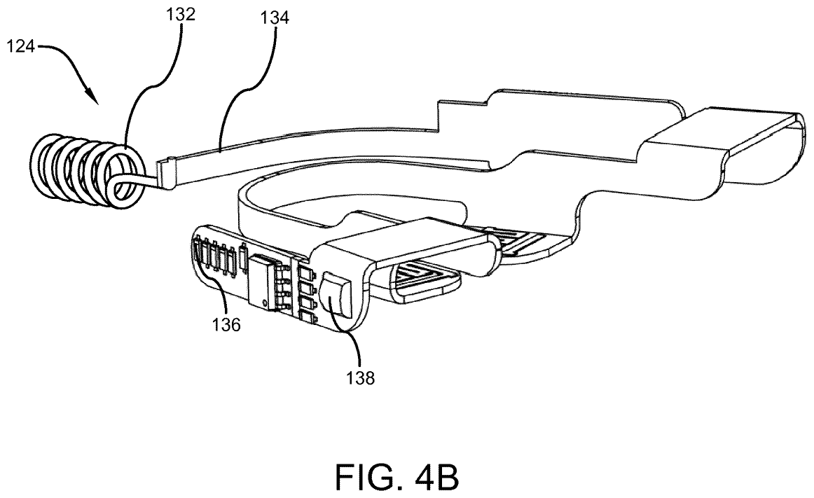

Referring now to FIGS. 4A-4B, a lower flex circuit 124 is detachably disposed in the lower silicone mouthpiece 104. The lower flex circuit 124 includes a microprocessor 126 and a plurality of computing modules 128 for providing processing of information captured from different sensors of the device 100. A second biosensor 130 is disposed on the inner horizontal surface 131 of the lower flex circuit 124. The second biosensor 130 also includes embedded EEG therein. A long-range antenna 132 is disposed on the proximal end 134 of the lower flex circuit 124 and is configured to extend the range of the Bluetooth module 118. The long-range antenna 132 has a distal end 133 that is adapted to detachably attach to the proximal end 134 of the lower flex circuit 124. For obstacle detection during functioning of the device 100, an infrared-based obstacle detection device 136 is disposed on the lower flex circuit 124. A power module is disposed on the lower flex circuit 124 for storing electrical power and regulating electrical power for operation of the device 100.

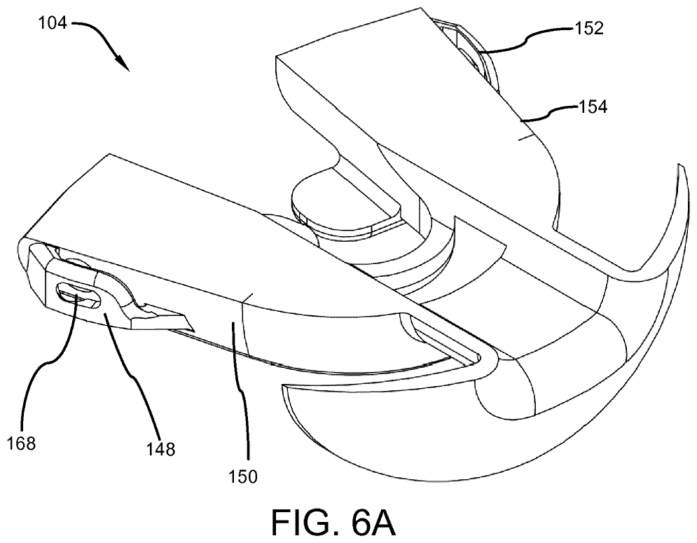

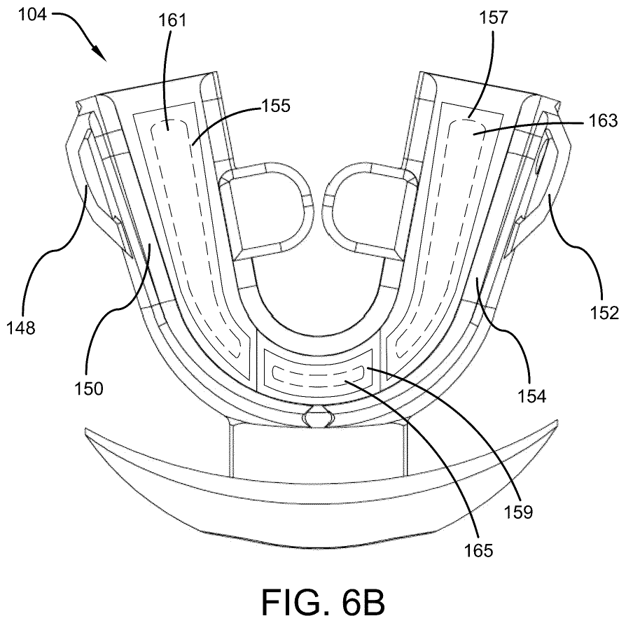

Referring now to FIGS. 1A-2, 6A-6B, and 14, the upper silicone mouthpiece 102 includes a first coupler accommodating protrusion 138 on a first arm 140 of the upper silicone mouthpiece 102. The upper mouthpiece 102 also includes a second coupler accommodating protrusion 142 on a second arm 144. The protrusions 138, 142 are disposed near the proximal end 146 of the upper silicone mouthpiece 102. A corresponding first coupler protrusion 148 is disposed on the first arm 150 of the lower silicone mouthpiece 104 and a corresponding second coupler protrusion 152 is disposed on the second arm 154. A first coupler 156 is detachably attached to the first coupler accommodating protrusion 138 and the corresponding first coupler protrusion 148 for operatively connecting the upper silicone mouthpiece 102 and the lower silicone mouthpiece 104. The first end 158 of the first coupler 156 is operatively connected to the first coupler accommodating protrusion 138 and the opposite end 160 of the first coupler 156 is operatively connected to the corresponding first coupler protrusion 148. The engaging members 162, 164 of the ends 158, 160 detachably engages with the apertures 166, 168 of the protrusions 138, 148. Referring to FIG. 6B, biochemical sensors can be mounted to the lower silicone mouthpiece 104. The first arm 150 and second arm 154 include neurochemical sensors 155, 157, 159 for absorbing saliva and blood traces into a chemical test-absorbent material which can synthesize ion protein for detecting brain tissue abnormalities. Once inserted into the mouth, the neurochemical sensors 155, 157, 159 trigger an autonomous neuro activity tracking, chemical synthesis, reporting, and predicting for diagnosing brain conditions. The sensors 155, 157, 159 can include gel soft tissue saliva/blood absorbers 161, 163, 165 and nano-synthesizers. The neurochemical sensors or biochemical sensors 155, 157, 159, and absorbers 161, 163, 165 can absorb amyloid and protein ions. The sensors 155, 157, 159 include micro-processors to store and remit data from the mouthguard device 100 to determine presence of neurological diseases.

The second coupler 170 is detachably attached to the second coupler accommodating protrusion 142 and the corresponding second coupler protrusion 152 for operatively connecting the upper silicone mouthpiece 102 and the lower silicone mouthpiece 104. The second coupler 170 has same structure and dimensions as of the first coupler 156 and is fastened in the same way as described for the first coupler 156. The couplers 156, 170 can be of different lengths to pivotally couple the upper silicone mouthpiece 102 and a lower silicone mouthpiece 104.

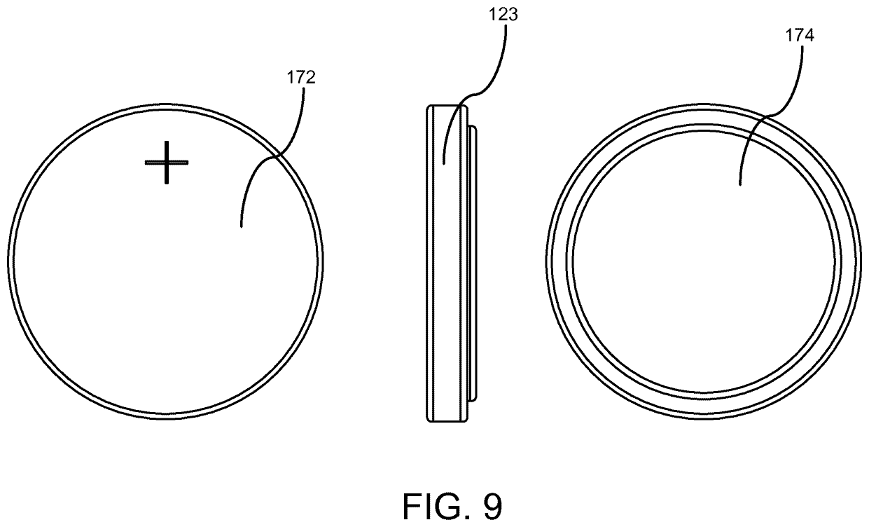

Referring to FIG. 9, the coin battery 123 used in the mouthguard device 100 is rechargeable and is replaceable. Further, the top surface 172 of the coin battery 123 preferably has positive polarity and the bottom surface 174 has negative polarity. The coin battery 123 can come in different sizes to fit mouthguard devices of different sizes.

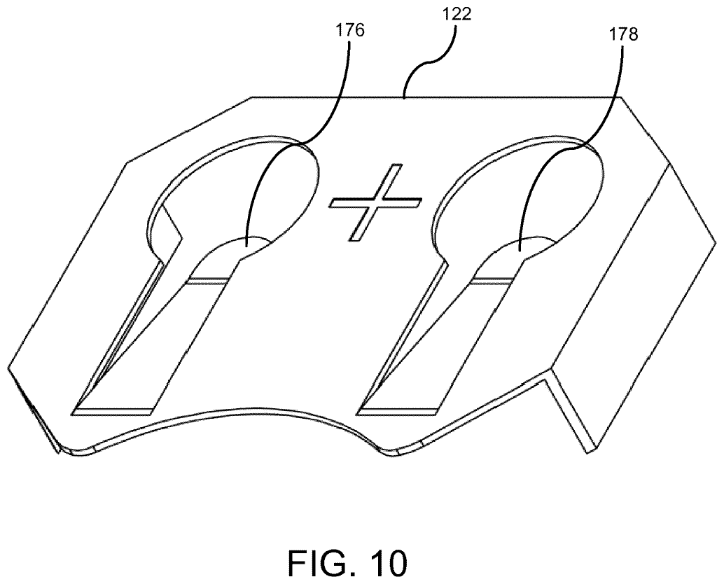

Referring to FIG. 10, the coin battery replaceable bracket 122 is adapted to support and secure the coin battery 123 while enabling the coin battery 123 to be operatively connected to the upper flex circuit 106 and the lower flex circuit 124. The bracket 122 is preferably made of a lightweight and insulating material and includes a pair of connectors 176, 178 for conducting and transferring electric power from the coin battery 123.

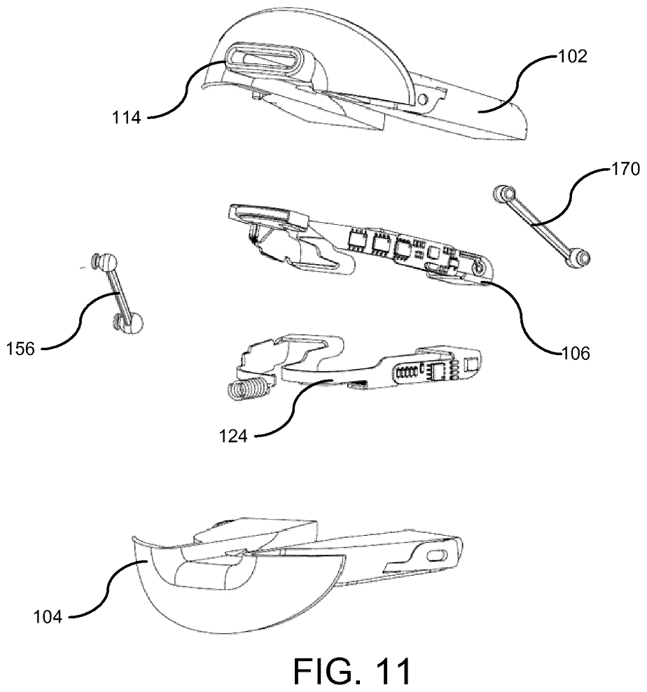

Referring now to FIG. 11, as illustrated in the exploded view, the upper mouthpiece 102 is positioned on the top flex circuit 106 and is adapted to accommodate the top flex circuit 106. The bracket 122 and the coin battery 123 are accommodated inside the battery cover 180 of the upper mouthpiece 102. The lower flex circuit 124 is positioned below the top flex circuit 106 and is protected and covered by the lower mouthpiece 104 from the bottom. The upper mouthpiece 102 and the lower mouthpiece 104 are secured pivotally using the couplers 156, 170 enabling the device 100 to have movement with jaw movement of wearer of the device while sealing the top flex circuit 106 and the lower flex circuit 124.

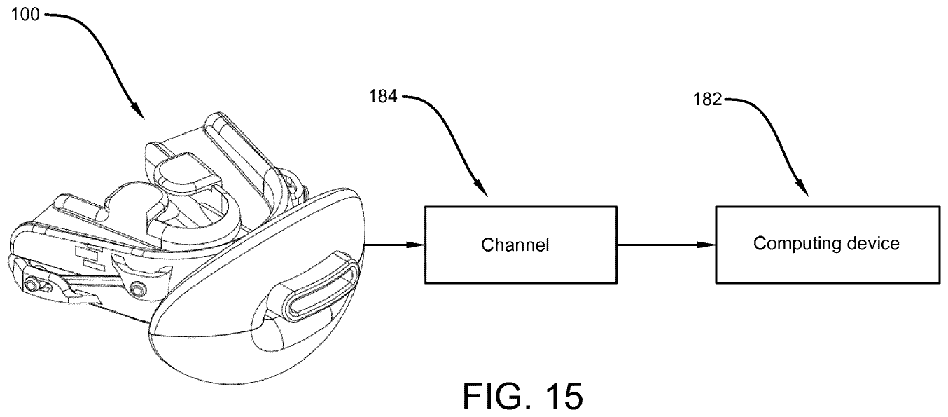

Referring now to FIG. 15, the mouthpiece device 100 is adapted to wirelessly couple to a programmed wireless device 182 which can be in the form of a smartphone, tablet, laptop, or any other computing system. The mouthpiece device 100 can send information in real-time or at a predetermined frequency. The wireless channel 184 is preferably Bluetooth but can be any short-range or long-range wireless communication channel. The transmission enables for immediate clinical intervention if needed. The programmed wireless device 182 can contain a software module or application which is adapted to synthesize digitized electroencephalograph into a metered accelerometer for clinical decision-making for injuries.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “malleable neurosensory mouthguard device”, “mouthguard device”, “sensor mouthguard device”, and “device” are interchangeable and refer to the malleable neurosensory mouthguard device 100 of the present invention.

Notwithstanding the forgoing, the malleable neurosensory mouthguard device 100 of the present invention can be of any suitable size and configuration as is known in the art without affecting the overall concept of the invention, provided that it accomplishes the above stated objectives. One of ordinary skill in the art will appreciate that the malleable neurosensory mouthguard device 100 as shown in the FIGS. are for illustrative purposes only, and that many other sizes and shapes of the malleable neurosensory mouthguard device 100 are well within the scope of the present disclosure. Although the dimensions the malleable neurosensory mouthguard device 100 are important design parameters for user convenience, the malleable neurosensory mouthguard device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A mouthguard device for monitoring head impacts comprising:
an upper mouthpiece portion;
a lower mouthpiece portion;
a flex circuit;
an accelerometer; and
a biosensor;
wherein said upper mouthpiece portion anatomically conforming to an upper jaw of a user;
wherein said upper mouthpiece portion having said flex circuit embedded therein;
wherein said flex circuit having a proximal end and a pair of side portions extending from said proximal end;
wherein said flex circuit having said accelerometer embedded in an outer surface of said flex circuit;
wherein said biosensor mounted to said flex circuit; and
further wherein said biosensor is an Electroencephalogram (EEG) for monitoring brain wave activity.

2. The mouthguard device for monitoring head impacts of claim 1, wherein said EEG is a Quantitative Electroencephalogram (QEEG).

3. The mouthguard device for monitoring head impacts of claim 1 further comprising a Bluetooth module for real-time communication between said mouthguard device and a remote electronic computing device.

4. The mouthguard device for monitoring head impacts of claim 3, wherein said remote electronic computing device is selected from the group consisting of a smartphone and a laptop.

5. The mouthguard device for monitoring head impacts of claim 1 further comprising an inertial measurement unit (IMU) chip mounted to said outer surface of said flex circuit.

6. The mouthguard device for monitoring head impacts of claim 5, wherein said proximal end having a replaceable bracket including a replaceable battery for providing electrical power to said mouthguard device.

7. The mouthguard device for monitoring head impacts of claim 5 further comprising another flex circuit embedded in said lower mouthpiece portion.

8. The mouthguard device for monitoring head impacts of claim 7, wherein said upper mouthpiece portion having a material selected from the group consisting of a silicone and a nylopolyethine.

9. The mouthguard device for monitoring head impacts of claim 8, wherein said lower mouthpiece portion having a material selected from the group consisting of a silicone and a nylopolyethine.

10. A mouthguard device for monitoring head impacts comprising:
an upper mouthpiece portion;
a lower mouthpiece portion;
a first flex circuit;
a first accelerometer;
a first biosensor;
a second flex circuit;
a second accelerometer; and
a second biosensor;
wherein said upper mouthpiece portion anatomically conforming to an upper jaw of a user;
wherein said upper mouthpiece portion having said first flex circuit embedded therein;
wherein said first flex circuit having a proximal end and a pair of side portions extending from said first proximal end;
wherein said first flex circuit having said first accelerometer embedded in an outer surface of said first flex circuit;
wherein said first biosensor mounted to said first flex circuit;
wherein said lower mouthpiece portion anatomically conforming to a lower jaw of the user;
wherein said lower mouthpiece portion having said second flex circuit embedded therein;
wherein said second flex circuit having a proximal end and a pair of side portions extending from said proximal end;
wherein said second flex circuit having said second accelerometer embedded in an outer surface of said second flex circuit;
wherein said second biosensor mounted to said second flex circuit; and
further wherein said first biosensor is an Electroencephalogram (EEG) for monitoring brain wave activity.

11. The mouthguard device for monitoring head impacts of claim 10, wherein said second biosensor is an Electroencephalogram (EEG) for monitoring brain wave activity.

12. The mouthguard device for monitoring head impacts of claim 10 further comprising a Bluetooth module for real-time communication between said mouthguard device and a remote electronic computing device.

13. The mouthguard device for monitoring head impacts of claim 12, wherein said remote electronic computing device is selected from the group consisting of a smartphone and a laptop.

14. The mouthguard device for monitoring head impacts of claim 10 further comprising an inertial measurement unit (IMU) chip mounted to said outer surface of one of said first flex circuit and said second flex circuit.

15. The mouthguard device for monitoring head impacts of claim 10, wherein said upper mouthpiece portion having a material selected from the group consisting of a silicone and a nylopolyethine.

16. The mouthguard device for monitoring head impacts of claim 15, wherein said lower mouthpiece portion having a material selected from the group consisting of a silicone and a nylopolyethine.

17. A mouthguard device for monitoring head impacts comprising:
an upper mouthpiece portion;
a lower mouthpiece portion;
a flex circuit;
an accelerometer;
a biochemical sensor; and
a coupler;
wherein said upper mouthpiece portion anatomically conforming to an upper jaw of a user;
wherein said upper mouthpiece portion having said flex circuit embedded therein;
wherein said flex circuit having a proximal end and a pair of side portions extending from said proximal end;
wherein said flex circuit having said accelerometer embedded in an outer surface of said flex circuit;
wherein said biochemical sensor mounted to at least one of said upper mouthpiece portion and said lower mouthpiece portion;
wherein said coupler pivotally couples said upper mouthpiece portion to said lower mouthpiece portion; and
further wherein said biochemical sensor is a blood and saliva absorber for absorbing amyloid and protein ions to detect presence of neurological diseases.

18. The mouthguard device for monitoring head impacts of claim 17 further comprising a Bluetooth module for real-time communication between said mouthguard device and a remote electronic computing device.

19. The mouthguard device for monitoring head impacts of claim 17 further comprising an inertial measurement unit (IMU) chip mounted to said outer surface of said flex circuit.

20. The mouthguard device for monitoring head impacts of claim 18 further comprising a long-range antenna mounted to said flex circuit for extending a range of said Bluetooth module.
